(19) United States
(12) Patent Application Publication
(10) Pub. No.: US 2011/0046703 A1
(43) Pub. Date: Feb. 24, 2011

(54) Heating device for low voltage thermal therapy

(76) Inventor: Chien-Chou Chen, Tainan County (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/461,596

(22) Filed: Aug. 18, 2009

(51) Int. Cl. A61F 7/08; H05B 3/34

(52) U.S. Cl. 607/99; 607/98; 607/108; 219/529

(57) ABSTRACT

This invention relates to a heating device for low voltage thermal therapy in which a heating layer is provided on the protector body and a lead wire is extended from the heating layer, a terminal being provided on the lead wire which is connected with the terminal of a thermostat. With this structure, low voltage direct current is supplied to the heating layer for generating heat required and temperature is regulated by the thermostat such that restoration effect on the injury condition of affected part is further enhanced.

### BACKGROUND OF THE INVENTION

1. Field of the Invention

This invention relates to a heating device for low voltage thermal therapy, especially to a heating device for low voltage thermal therapy in which a heating layer is provided on the protector body and the heating layer is connected to a thermostat that is, in turn, connected with a power supply. In this manner, low voltage direct current is supplied to the heating layer for generating heat required and temperature is regulated by the thermostat such that restoration effect on the injury condition of affected part is further enhanced.

2. Brief Description of Prior Art

The main function of a protective means such as waist brace, knee brace, shoulder brace is to provide support and compression action for wounded or unwounded joint, tendon, ligament so as to avoid injury resulted from over-exercise or violent motion, or to avoid further enlargement of injury by the support action provided by waist brace, knee brace, shoulder brace, or to avoid further dilation of swollen part and to allow the muscle to be in tensioned state by the stretch taut, which is generated by the compression action, conducted on the affected part such that further injury can be prevented.

However, ordinary waist brace, knee brace, shoulder brace can only provide support and compression effect. If a hot compress is further conducted so as to facilitate blood circulation on affected part, the effect of stretching the tendon and stimulating the blood circulation can be achieved so that restoration on the injury condition of affected part can be facilitated.

Referring to U.S. Pat. No. 5,432,322 entitled “Electric Heating Pad”, the pad body includes an heater unit interposed therein which is made of a conductor formed by printing between two layers of plastic sheet, and the heater unit is in turn connected with a power cord extended from the protector body, the end portion of the power cord is provided with a plug for the plug-in with a socket of commercial alternating current. After the plug-in of the plug with the socket, alternating current flows into the heater unit which then converts the electricity into heat energy, the heat energy in turn penetrating out through the pad body to conduct hot compress on the affected part of user. However, this design is found to have the following shortcomings in practical implementation:

1. In as much as the heater unit is made of a conductor formed by printing between two layers of plastic sheet, the heater unit itself has considerable hardness which make the heater unit difficult to conform itself to the body contour of a variety of users. Thus, this will result in bad effect of hot compress on the injury condition of affected part.

2. In as much as the power cord of the prior art is arranged to extend out from the pad body after connection with the heater unit, the power cord cannot be removed when the pad body needs cleaning for removing the dirty. Thus, this will cause inconvenience in cleaning and may cause water infiltration into breakage, if any, so that danger of accidental fire triggered by short circuit on wires might be happened when the power cord is again in plug with the power source.

3. The prior art directly utilizes alternating current to supply the power required for energizing the heater unit. However, alternating current is so unstable as to cause the damage of the heater unit easily.

4. The protector body in this prior art is of plane shape which fails to conduct close-contact or winding on user's shoulder or knee by perfect conformation to the joint of shoulder or knee. Thus, this will cause imperfect hot compress effect.

Seeing the above shortcomings that conventional protective means only possesses support and compression effect and that the “Electric Heating Pad” unveiled in U.S. Pat. No. 5,432,322 has the defects of inability to conform itself to the outside contour of users for close-contact or winding on user's body and of difficulty in cleaning, inventor of the present invention conducts research and improvement on the existing structure based on his abundant experience of R&D and manufacturing in relevant field and diversified skillful contemplation so as to attain better performance in practical application.

### SUMMARY OF THE INVENTION

The present invention relates to a heating device for low voltage thermal therapy. The main object of its implementation is essentially to provide a protector in which direct current of low voltage is supplied to a heating layer disposed therein for generating heat so as to produce hot compress effect.

In order to achieve above object, the heating device for low voltage thermal therapy proposed by inventor of the present invention comprises:

a protector body;

a heating layer assembled in the inner surface of the protector body, essentially including a high-impedance wire, a lead wire connected with the high-impedance wire, and a shielding layer overlaid on the outside of the high-impedance wire and the lead wire, the lead wire being extended to the outside of the shielding layer, the end portions of the lead wire being provided with a terminal;

a thermostat, having a thermostat main body provided with a temperature control switch, a temperature control button, the temperature control switch and temperature control button being in connection with the temperature control element built in the thermostat main body, lead wires connecting with the temperature control element being extended from both sides of the thermostat main body, the end portions of two lead wires being formed with terminals respectively so as to connect with the terminal provided on end portion of the lead wire of the heating layer and the terminal provided on end portion of the lead wire of the power supply respectively;

a power supply, including a power supply main body and a lead wire, an AC-DC converting circuit being provided in the power supply main body, a plug being provided on and protruded out of the power supply main body so as to be connected with the AC-DC converting circuit which is further connected to the lead wire extended from the power supply main body, and terminal being provided on the end portion of the lead wire;

With this structure, low voltage DC is utilized to supply the heating layer with the required power for generating heat so as to produce hot compress effect on affected part. Furthermore, the electric current delivered from the power supply is adjusted by the thermostat so that the power supplied to the heating layer is regulated to control the temperature of hot compress. In this manner, the restoration effect on the injury condition of affected part can be effectively enhanced.

### BRIEF DESCRIPTION OF THE DRAWINGS

FIG. 1 is a perspective view showing the entirety of the present invention.

FIG. 2 is a perspective exploded view showing the protector body in waist brace type of the present invention.

FIG. 3 is a perspective exploded view showing the protector body in shoulder brace type of the present invention.

FIG. 4 is a perspective exploded view showing the protector body in knee brace type of the present invention.

FIG. 5 is a view showing the assembly of the protector body in waist brace type of the present invention.

FIG. 6 is a view showing the assembly of the protector body in shoulder brace type of the present invention.

FIG. 7 is a view showing the assembly of the protector body in knee brace type of the present invention.

FIG. 8 is a sectional view of the present invention.

FIG. 9 is a perspective view showing the heating layer of the present invention.

### DETAILED DESCRIPTION OF PREFERRED EMBODIMENTS

The technical content, object and effectiveness of the present invention will become more apparent by the detailed description of preferred embodiments of the present invention in conjunction with the accompanying drawings.

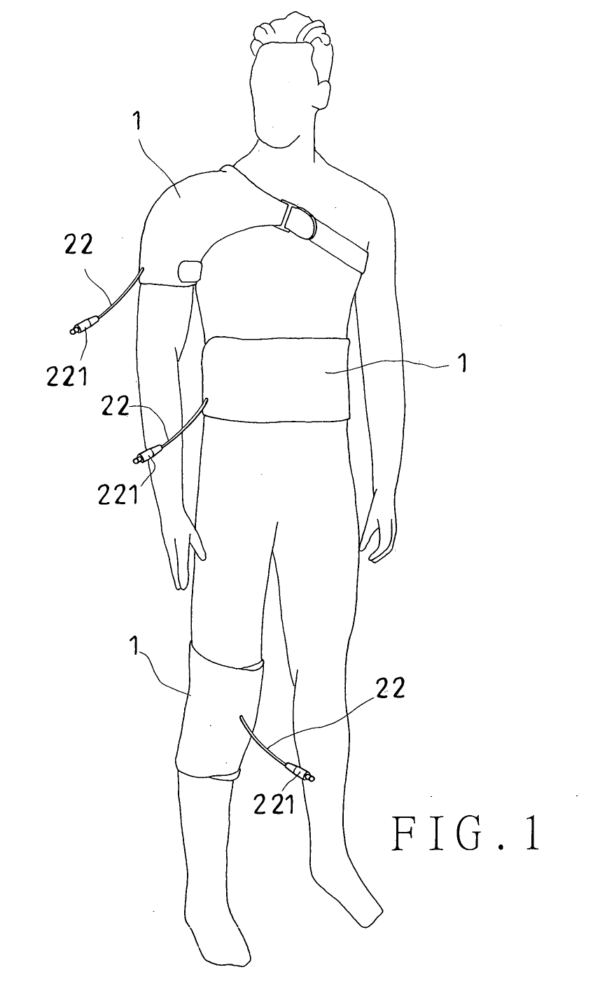

Firstly referring to FIG. 1, the heating device for low voltage thermal therapy of the present invention comprises a protector body (1), a heating layer (2), a thermostat (3) and a power supply (4).

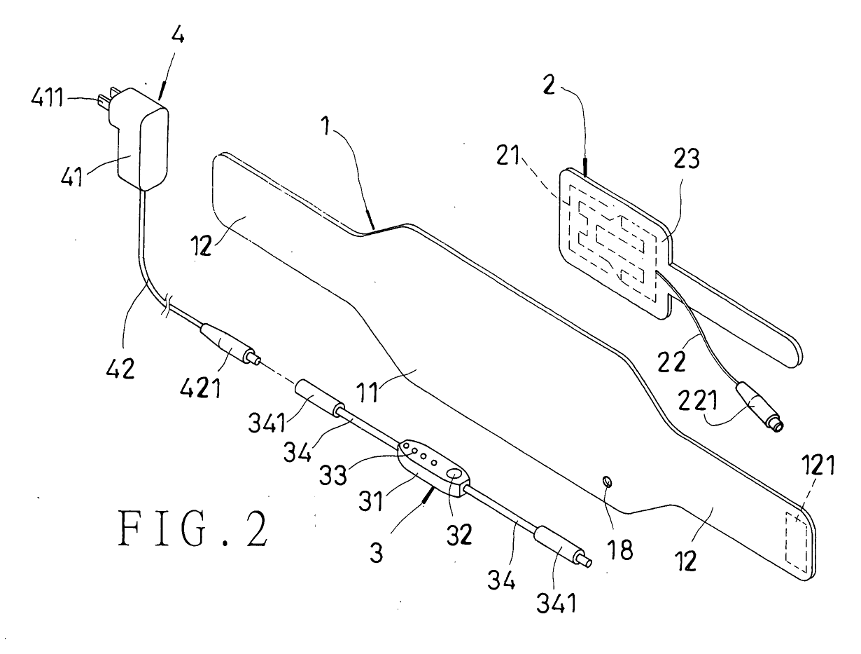

The protector body (1) can be formed in the type of waist brace, shoulder brace and knee brace. Taking the formation of waist brace as an example, as shown in FIG. 2, the protector body (1) is formed as a rectangular portion (11) corresponding to the waist portion of human body and two binding girdles (12) are extended at both sides of the rectangular portion (11), a fastening member (121) being provided on one of the binding girdles (12). In this manner, a waist brace is formed.

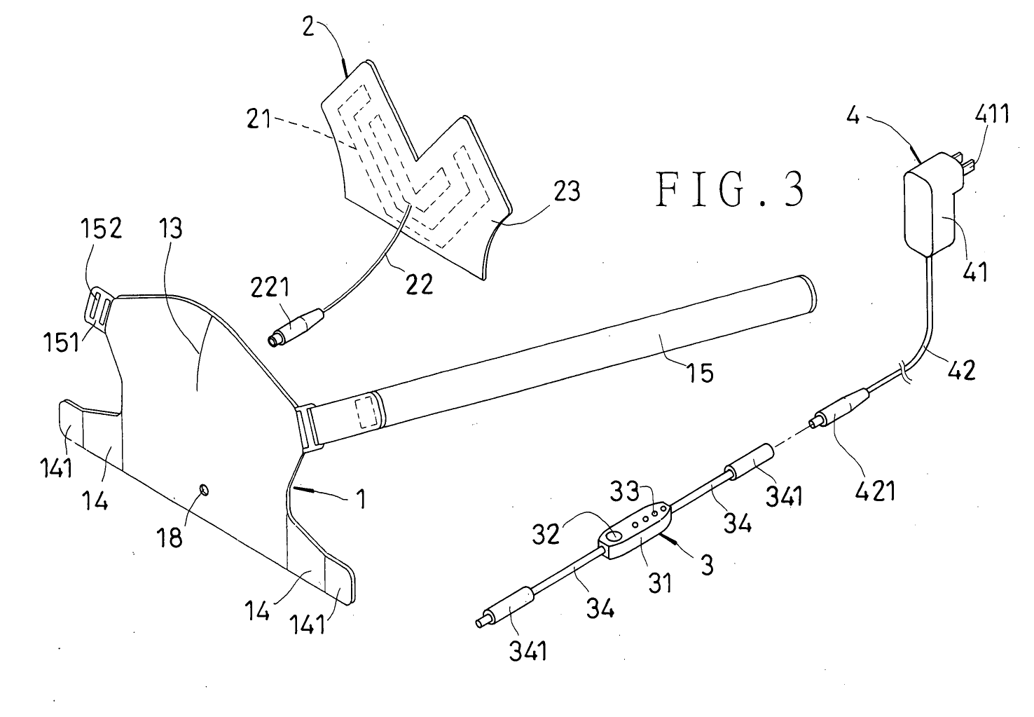

Referring to FIG. 3, the protector body (1) is alternatively formed as a concaved portion (13) corresponding to the shoulder of human body. Two binding girdles (14) are extended at both sides of the lower edge of the concaved portion (13), the end portions of the binding girdles (14) being provided with fastening members (141), a fastening belt (15) being connected at one side of the upper edge of the concaved portion (13), a buckle member (151) being provided at the other side of the upper edge of the concaved portion (13) and buckle holes (152) being provided on the buckle member (151) so that the fastening belt (15) can pass through the buckle holes (152) and buckles in the buckle member (151). In this manner, a shoulder brace is thus formed.

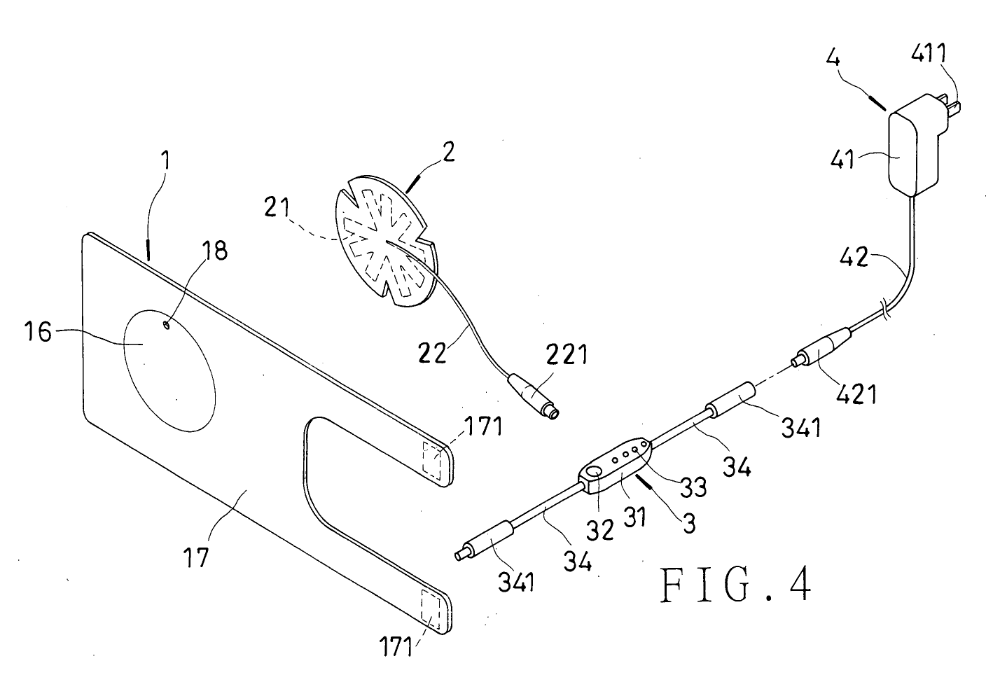

Referring to FIG. 4, the protector body (1) is further alternatively formed as a semi-spherical portion (16) at one side, which covers on the corresponding knee of user. Two binding girdles (17) are extended at both sides of the semi-spherical portion (16), the end portions of the binding girdles (17) being provided with fastening members (171). In this manner, a knee brace is thus formed.

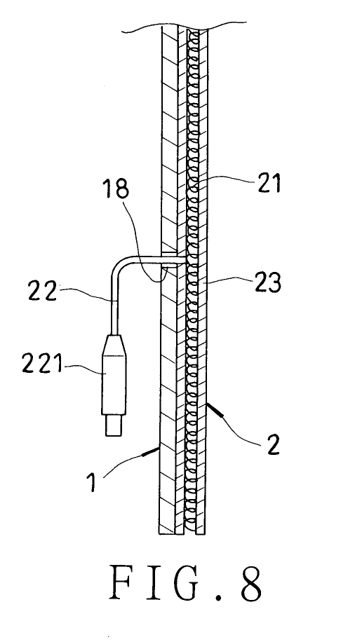

Further, an aperture (18) is formed on the protector body (1) for the lead wire (22) to go outside, as shown in FIG. 8.

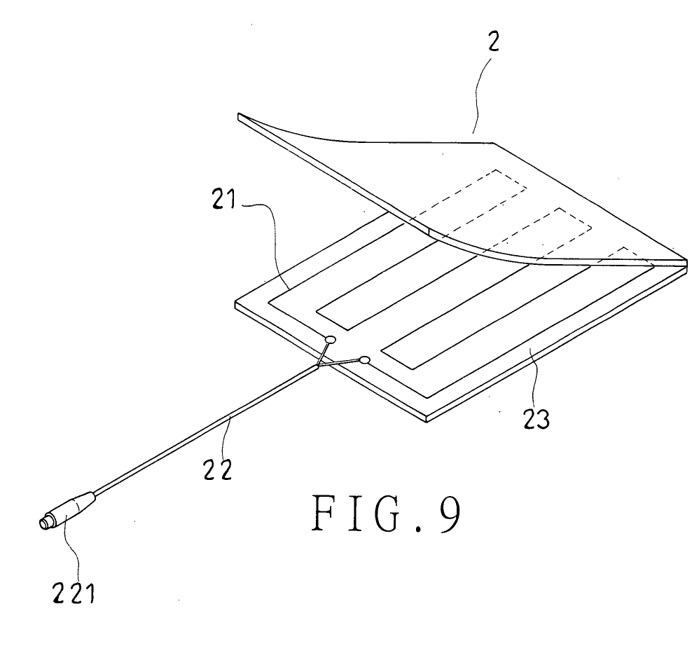

The heating layer (2), as shown in FIG. 9, is assembled on the inner surface of the protector body (1), which essentially includes a high-impedance wire (21) which is carbon fiber wire in this case, a lead wire (22) connected with the high-impedance wire (21), and a fabric shielding layer (23) wrapped on the outside of the high-impedance wire (21) and the lead wire (22). The lead wire (22) extends to the outside of the shielding layer (23) and a terminal (221) is provided on the end portion of the lead wire (22).

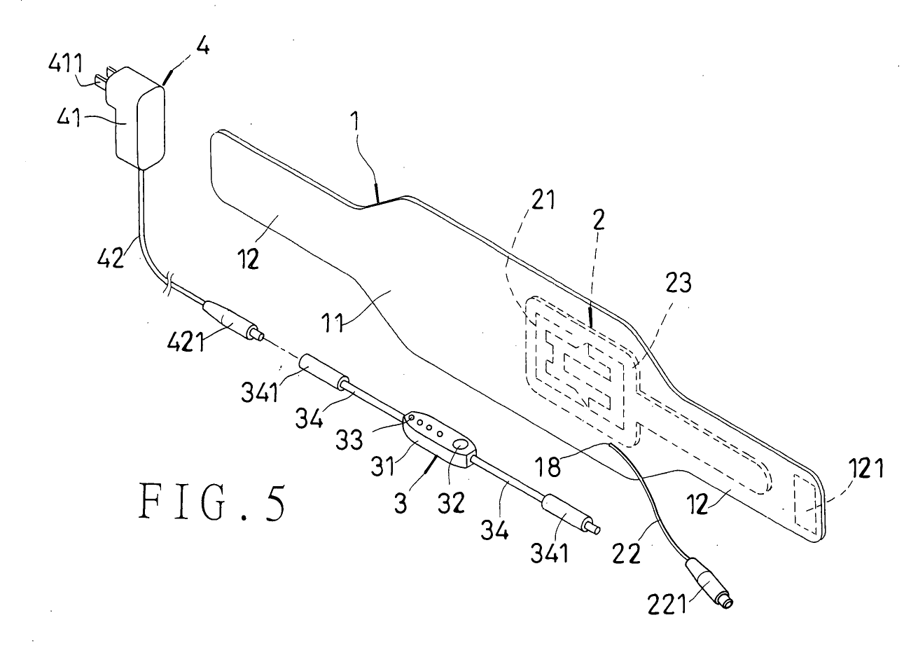

In the protector body (1) formed as a waist brace, the heating layer (2) is formed as a rectangular sheet body so as to be installed on the protector body (1) formed with a rectangular portion (11), as shown in FIG. 5.

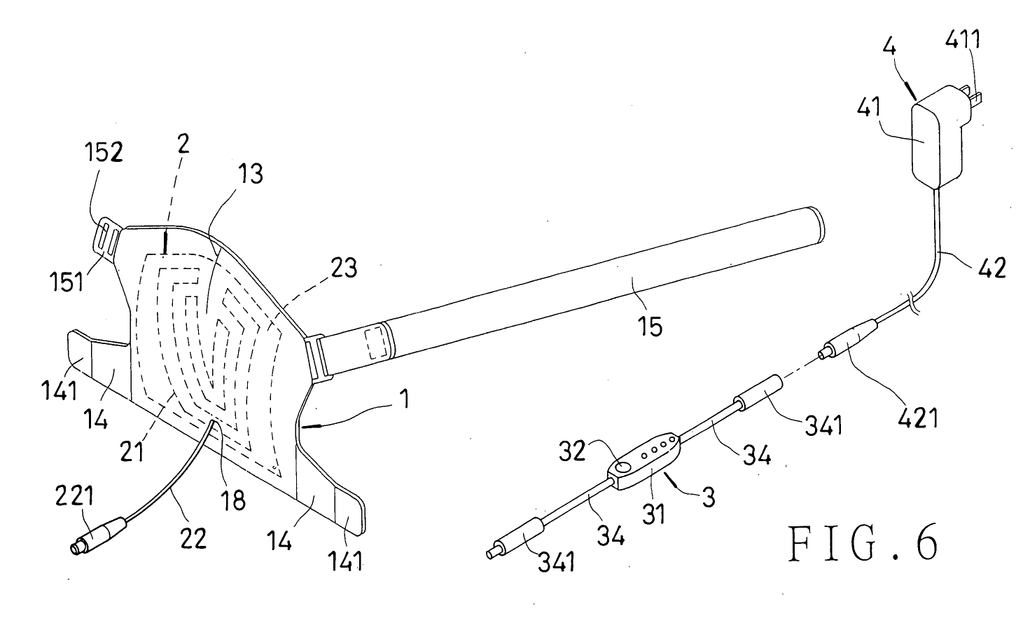

Alternatively, in the protector body (1) formed as a shoulder brace, the heating layer (2) is formed as a polygonal sheet body formed by the combination of two arc-shape sheet body such that the heating layer (2) of polygonal sheet body is correspondingly installed and overlaid on the protector body (1) which is formed as a concaved portion (13), as shown in FIG. 6.

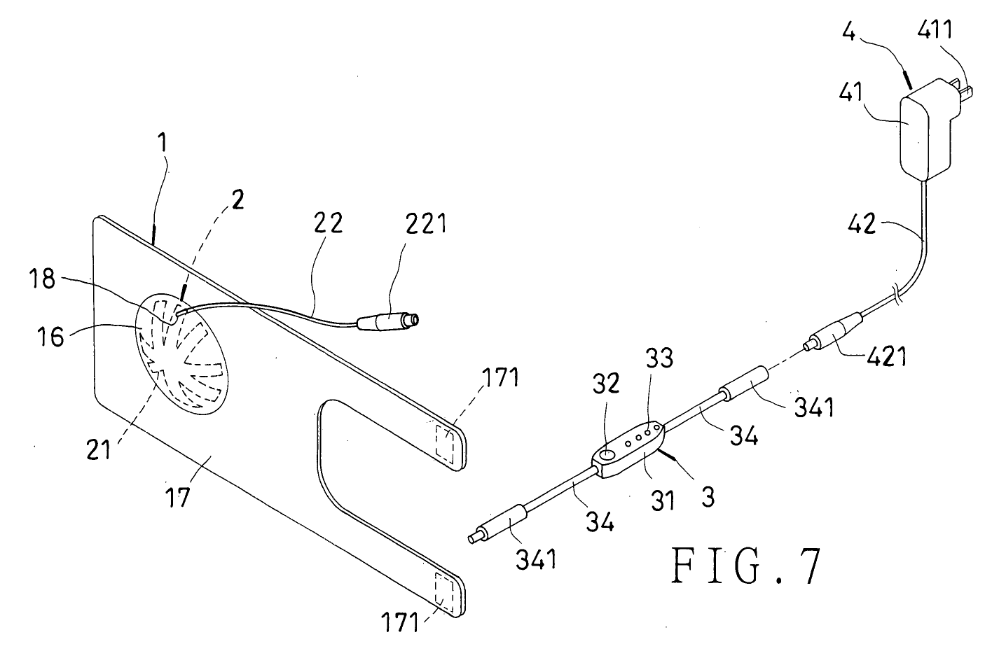

Further, in the protector body (1) formed as a knee brace, the heating layer (2) is formed as a circular sheet body on which several slits are provided for mounting the heating layer (2) of circular sheet body within the protector body (1) formed with a semi-spherical portion (16), as shown in FIG. 7.

The thermostat (3) has a thermostat main body (31) which is provided with a temperature control switch (32), a temperature control button (33) and a temperature display unit thereon. The temperature control switch (32), the temperature control button (33) and the temperature display unit are connected with the temperature control element, which is for example a temperature control resistor, provided in the thermostat main body (31). Two lead wires (34) connected with the temperature control element are extended at both sides of the thermostat main body (31), and terminals (341) are provided respectively at the end portions of the two lead wires (34).

The power supply (4) includes a power supply main body (41) and a lead wire (42). An AC-DC converter circuit is provided in the power supply main body (41) and a plug (411) contacted with the AC-DC converter circuit is protruded from the power supply main body (41). Furthermore, the AC-DC converter circuit is connected with the lead wire (42) extended from the power supply main body (41) and a terminal (421) is provided at the end portion of the lead wire (42).

When putting into practice with this structure, as shown in FIGS. 1 and 5, user can choose suitable protective means according to wounded portion on user's waist, knee or shoulder. For example, a waist brace formed as a rectangular portion (11) can be chosen for a wounded part on waist portion such that big area of waist portion can be wound by the waist brace first and is tied to fix by the binding girdles (12).

In turn, the terminal (221) provided on the end portion of the lead wire (22) of the heating layer (2) is connected to the terminal (341) fixed on the end portion of one lead wire (34) of the thermostat (3), then the terminal (341) fixed on the end portion of the other one lead wire (34) of the thermostat (3) is coupled to the terminal (421) formed on the end portion of the lead wire (42) of the power supply (4). Subsequently, the plug (411) of the power supply (4) is in plug with the socket connected with commercial utility power.

In this manner, the commercial utility power (110˜240 volts) is converted into low voltage DC of 12 volts by the AC-DC converter circuit provided in the power supply (4), and the DC thus converted is then supplied to the heating layer (2) through the thermostat (3) in ON state so that the high-impedance wire (21), which is carbon fiber in this case, in the heating layer (2) is energized to generate heat. Thus, hot compress effect can be conducted on affected portion of user.

In turn, when user finds the overheat or over-low in the temperature of the heating layer (2), the temperature control button (33) of the thermostat (3) can be adjusted to regulate the temperature, and the state of temperature adjustment can be clearly seen from the temperature display unit so as to ensure the conduction of temperature control. In this manner, user can feel more comfortable by the control and regulation on temperature, and the rate of restoration on the injury condition of affected part can be further enhanced.

Furthermore, the flexibility of the high-impedance wire (21), which is carbon fiber in this case, provided in the interior of the heating layer (2) enables the protector body (1) conform to the external contour of each user so as to form excellent close-contact state with the affected portion of user. Therefore, the effect of stimulating blood circulation on affected part and enhancing restoration on injury part of affected portion can be achieved.

Moreover, referring to FIG. 6, a protector body (1) formed with concaved portion (13) can be used for the wounded part on shoulder. In this case, the concaved portion (13) is directed to the shoulder of human body and then the binding girdles (14) provided on the lower edge are tied together by the fasteners (141) so as to be fixed on the arm of human body. Further, the fastening belt (15) connected on the upper edge of the concaved portion (13) is allowed to pass through the buckle holes (152) and to buckle in the buckle member (151) provided on the other side of the upper edge of the concaved portion (13), after it is wound in forward direction around the shoulder and arm of the other side. In this manner, the protector body (1) formed as shoulder brace can be stably fixed on the shoulder of human body so as to conduct hot compress on the affected portion of shoulder.

Further referring to FIG. 7, a protector body (1) formed with a semi-spherical portion (16) is selected to be used on wounded portion of knee. In this case, the semi-spherical portion (16) is used to cover the knee portion of human body and two binding girdles (17) connected and extended from the semi-spherical portion (16) are tied together by the fasteners provided at the end portions. In this manner, the protector body (1) formed as a knee brace is fixed on the knee portion of human body so that therapeutic effect of hot compress can be conducted on the knee portion.

Based on the foregoing structure and embodiment, the heating device for low voltage thermal therapy of the present invention has the following advantages:

1. The protector body in the present invention is formed as waist brace, shoulder brace and knee brace corresponding to the waist portion, shoulder portion and knee portion in such a manner that compression and support actions can be conducted on the waist portion, shoulder portion and knee portion of human body. Furthermore, the heating layer provided in the protector body is utilized to conduct hot compress on the affected portion so as to achieve the effect of facilitating blood circulation on affected part and stretching the tendon and stimulating the blood circulation. Moreover, the restoration effect on the injury condition of affected part can be enhanced.

2. A power supply having AC-DC converter is provided in the present invention. Thus, AC can be converted into low voltage DC having stable feature so as to be supplied for use in thermostat and heating layer. In this manner, the effectiveness of preventing the thermostat and the heating layer from damage can be achieved.

3. A thermostat is further provided between the power supply and the heating layer in the present invention. The electric current supplied from the power supply is adjusted by the control button provided on the thermostat so as to further control the heat generated by the high-impedance wire in the heating layer. In this manner, when user finds the overheat or over-low in the temperature of the heating layer, the temperature control button of the thermostat can be adjusted to make user feel more comfortable and to further enhance the restoration on the injury condition of affected part.

4. In the present invention, a temperature display unit is further provided on the thermostat. User can see the temperature adjustment state clearly from the temperature display unit so as to ensure the conduction of the regulation and control on temperature.

5. A high-impedance wire, which is carbon fiber in this case, is provided within the heating layer in the present invention. The flexibility of the high-impedance wire enables the protector body conform to various external contours of users so as to form excellent close-contact state with the affected portion of users. Therefore, the effect of stimulating blood circulation on affected part and enhancing restoration on injury part of affected portion can be achieved.

6. A lead wire is connected and extended from the heating layer, and a terminal is provided on the end portion of the lead wire so as to connect freely with the terminal on end portion of the lead wire of the thermostat. In this manner, when the protector becomes dirty, the mutually connected terminals of the heating layer and the thermostat can be detached apart so as to facilitate the cleaning of the protector body.

7. In the present invention, a concaved portion and a semi-spherical portion are correspondingly formed according to the position of shoulder portion and knee portion of human body. With this structure, excellent overlying and close-contact with the joints of shoulder portion and knee portion can be achieved so as to achieve hot compress and restoration on wounded part.

Summing up above, the above embodiments of this invention can reach expected effectiveness, and the specific structure disclosed herein has yet not seen in the prior art of the same category of product, even has not been opened to the public before application.

What is claimed is:

1. A heating device for low voltage thermal therapy, comprising:
a protector body having an aperture formed thereon for the lead wire of the heating layer to go outside;
a heating layer assembled in the inner surface of the protector body, essentially including a high-impedance wire, a lead wire connected with the high-impedance wire, and a shielding layer overlaid on the outside of the high-impedance wire and lead wire, and the lead wire being extended to the outside of the shielding layer, the end portion of the lead wire being provided with a terminal.

2. A heating device for low voltage thermal therapy as claimed in claim 1, further comprising a thermostat having a thermostat main body provided with a temperature control switch, a temperature control button, the temperature control switch and the temperature control button being in connection with the temperature control element built in the thermostat main body, two lead wires connecting with the temperature control element being extended from both sides of the thermostat main body, the end portions of two lead wires being formed with terminals respectively so as to connect with the terminal provided on the end portion of the lead wire of the heating layer and the terminal provided on the end portion of the lead wire of a power supply respectively;
said power supply including a power supply main body and a lead wire, an AC-DC converting circuit being provided in the power supply main body, a plug being provided on and protruded out of the power supply main body so as to be connected with the AC-DC converting circuit which is further connected to the lead wires extended from the power supply main body, and terminal being provided on the end portion of the lead wire.

3. A heating device for low voltage thermal therapy as claimed in claim 2, wherein said thermostat is further provided a temperature display unit on the thermostat main body.

4. A heating device for low voltage thermal therapy as claimed in claim 1, wherein said protector body is formed as a waist brace which is formed with a rectangular portion, two binding girdles being connected and extended at both sides of said rectangular portion, a fastening member being provided on the end portion of one binding girdles, said heating layer being formed correspondingly as a rectangular sheet body so as to be installed at the rectangular portion formed on said protector body.

5. A heating device for low voltage thermal therapy as claimed in claim 1, wherein said protector body is formed as a shoulder brace which has a concaved portion corresponding to the shoulder portion of human body, two binding girdles being connected and extended from both sides the lower edge of the concaved portion, the end portions of the binding girdles being provided with fastening members, a fastening belt being connected at one side of the upper edge of the concaved portion, a buckle member being provided at the other side of the upper edge of the concaved portion and buckle holes being provided on the buckle member so that the fastening belt can pass through the buckle holes and buckle in the buckle member, said heating layer being formed as a polygonal sheet body formed by the combination of two arc shape sheet body such that said heating layer of polygonal sheet body is correspondingly installed on the protector body formed with a concaved portion.

6. A heating device for low voltage thermal therapy as claimed in claim 1, wherein said protector body is formed as a knee brace, a semi-spherical portion corresponding to the shoulder portion of human body being formed at one side of said protector body, two binding girdles being connected and extended at both sides of the semi-spherical portion, the end portions of the binding girdles being provided with fastening members, said heating layer being formed as a circular sheet body which has several slits formed thereon so as to be installed on the semi-spherical portion of the protector body.

7. A heating device for low voltage thermal therapy as claimed in claim 1, wherein said high-impedance wire is a wire of carbon fiber.
